(19) United States
(12) Patent Application Publication
(10) Pub. No.: US 2018/0368770 A1
(43) Pub. Date: Dec. 27, 2018

(54) CAPACITIVE SENSOR FOR ASSESSING CUFF APPLICATION

(71) Applicant: Edwards Lifesciences Corporation, Irvine, CA (US)

(72) Inventors: Olaf Schraa, Amsterdam (NL); Christianus Joannes Jozef Eugène van Groeningen, Utrecht (NL); Berend Westerhof, Amstelveen (NL)

(21) Appl. No.: 16/003,511

(22) Filed: Jun. 8, 2018

Related U.S. Application Data
(60) Provisional application No. 62/525,023, filed on Jun. 26, 2017.

(51) Int. Cl. A61B 5/00; A61B 5/022; G01L 5/04

(52) U.S. Cl. CPC A61B 5/6843; G01L 5/045; A61B 5/02241

(57) ABSTRACT

Disclosed is a method for assessing application and/or tightness of a cuff, comprising: determining a capacitance between a conducting component embedded in the cuff and earth, wherein the conducting component is electrically insulated from earth and from a patient; and determining application and/or tightness of the cuff based on the determined capacitance between the conducting component embedded in the cuff and earth.

### CROSS-REFERENCE TO RELATED APPLICATIONS

This applications claims priority to U.S. Provisional Patent Application No. 62/525,023, filed Jun. 26, 2017, the contents of which is incorporated herein in its entirety.

### BACKGROUND

### Field

Embodiments of the invention relate to non-invasive blood pressure measurement, and more particularly, to assessment of application and/or tightness of a finger, arm, or leg cuff used in non-invasive blood pressure measurement.

### Relevant Background

Volume clamping is a technique for non-invasively measuring blood pressure in which pressure is applied to a subject's finger in such a manner that arterial pressure may be balanced by a time varying pressure to maintain a constant arterial volume. In a properly fitted and calibrated system, the applied time varying pressure is equal to the arterial blood pressure in the finger. The applied time varying pressure may be measured to provide a reading of the patient's arterial blood pressure. Clamping techniques may also be used for other body parts, such as, arms, legs, etc.

A known method to obtain an indication of the tightness of the cuff application involves measuring for example the pressure response to quick inflation. In the case of the volume clamp technology, this can only be done when there is not a blood pressure measurement going on. Also, since during volume clamp measurements on the finger, the volume of the finger under the cuff decreases slowly as blood and interstitial fluids are pressed away, the tightness of the cuff changes and the cuff may become too loose. Measurements have shown that if the circumference of the finger is decreased by 3%, it affects reported blood pressure values substantially. This change in volume is especially the case in subjects with edema, in pregnant women, or when long term (e.g., 8 hour) measurements are made on the same finger.

Because the cuff is connected via a tube to the pressure generator, the resistance of the tube limits assessment of cuff volume based on the pressure response, such that: the measured response for large cuff volumes is almost completely determined by the resistance of the tube (and not the cuff volume). Therefore, the known method for assessing cuff tightness based on the pressure response as described above may be unreliable in certain circumstances.

### SUMMARY

Embodiments of the invention may relate to a method for assessing application and/or tightness of a cuff, comprising: determining a capacitance between a conducting component embedded in the cuff and earth, wherein the conducting component is electrically insulated from earth and from a patient; and determining application and/or tightness of the cuff based on the determined capacitance between the conducting component embedded in the cuff and earth.

### BRIEF DESCRIPTION OF THE DRAWINGS

FIG. 1 is a diagram of an example of a blood pressure measurement system.

FIG. 2 is a block diagram illustrating example control circuitry.

FIG. 3 is a diagram illustrating an example cuff application/tightness assessment module.

FIG. 4 is a diagram illustrating an example cuff application/tightness assessment module implemented in a flexible printed circuit.

FIG. 5 is a flowchart illustrating an example method for assessing application and/or tightness of a cuff.

### DETAILED DESCRIPTION

Embodiments of the invention may relate to a method for assessing application and/or tightness of a cuff, comprising: determining a capacitance between a conducting component embedded in the cuff and earth, wherein the conducting component is electrically insulated from earth and from a patient; and determining application and/or tightness of the cuff based on the determined capacitance between the conducting component embedded in the cuff and earth.

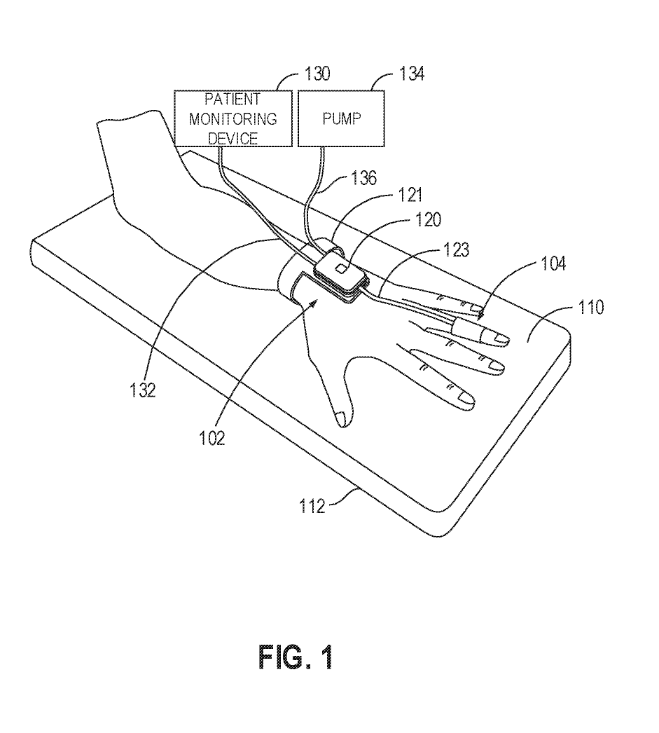

With reference to FIG. 1, an example of an environment in which a finger cuff 104 may be implemented will be described. As an example, a blood pressure measurement system 102 that includes a finger cuff 104 that may be attached to a patient's finger and a blood pressure measurement controller 120 that may be attached to the patient's body (e.g., a patient's wrist or hand) is shown. The blood pressure measurement system 102 may further be connected to a patient monitoring device 130, and, in some embodiments, a pump 134. Further, finger cuff 104 may include a bladder (not shown) and an light-emitting diode (LED)-photodiode (PD) pair (not shown), which are conventional for finger cuffs.

In one embodiment, the blood pressure measurement system 102 may include a pressure measurement controller 120 that includes: a small internal pump, a small internal valve, a pressure sensor, and control circuitry. In this embodiment, the control circuitry may be configured to: control the pneumatic pressure applied by the internal pump to the bladder of the finger cuff 104 to replicate the patient's blood pressure based upon measuring the pleth signal received from the LED-PD pair of the finger cuff 104. Further, the control circuitry may be configured to: control the opening of the internal valve to release pneumatic pressure from the bladder; or the internal valve may simply be an orifice that is not controlled. Additionally, the control circuitry may be configured to: measure the patient's blood pressure by monitoring the pressure of the bladder based upon the input from a pressure sensor, which should be the same as patient's blood pressure, and may display the patient's blood pressure on the patient monitoring device 130.

In another embodiment, a conventional pressure generating and regulating system may be utilized, in which, a pump 134 is located remotely from the body of the patient. In this embodiment, the blood pressure measurement controller 120 receives pneumatic pressure from remote pump 134 through tube 136 and passes on the pneumatic pressure through tube 123 to the bladder of finger cuff 104. Blood pressure measurement device controller 120 may also control the pneumatic pressure (e.g., utilizing a controllable valve) applied to the finger cuff 104 as well as other functions. In this example, the pneumatic pressure applied by the pump 134 to the bladder of finger cuff 104 to replicate the patient's blood pressure based upon measuring the pleth signal received from the LED-PD pair of the finger cuff 104 and measuring the patient's blood pressure by monitoring the pressure of the bladder may be controlled by the blood pressure measurement controller 120 and/or a remote computing device and/or the pump 134 and/or the patient monitoring device 130. In some embodiments, a blood pressure measurement controller 120 is not used at all and there is simply a connection from the tube 123 to the finger cuff 104 from a remote pump 134 including a remote pressure regulatory system, and all processing for the pressure generating and regulatory system, data processing, and display is performed by a remote computing device.

Continuing with this example, as shown in FIG. 1, a patient's hand may be placed on the face 110 of an arm rest 112 for measuring a patient's blood pressure with the blood pressure measurement system 102. The blood pressure measurement controller 120 of the blood pressure measurement system 102 may be coupled to a bladder of the finger cuff 104 in order to provide pneumatic pressure to the bladder for use in blood pressure measurement. Blood pressure measurement controller 120 may be coupled to the patient monitoring device 130 through a power/data cable 132. Also, in one embodiment, as previously described, in a remote implementation, blood pressure measurement controller 120 may be coupled to a remote pump 134 through tube 136 to receive pneumatic pressure for the bladder of the finger cuff 104. The patient monitoring device 130 may be any type of medical electronic device that may read, collect, process, display, etc., physiological readings/data of a patient including blood pressure, as well as any other suitable physiological patient readings. Accordingly, power/data cable 132 may transmit data to and from patient monitoring device 130 and also may provide power from the patient monitoring device 130 to the blood pressure measurement controller 120 and finger cuff 104.

As can be seen in FIG. 1, in one example, the finger cuff 104 may be attached to a patient's finger and the blood pressure measurement controller 120 may be attached on the patient's hand or wrist with an attachment bracelet 121 that wraps around the patient's wrist or hand. The attachment bracelet 121 may be metal, plastic, Velcro, etc. It should be appreciated that this is just one example of attaching a blood pressure measurement controller 120 and that any suitable way of attaching a blood pressure measurement controller to a patient's body or in close proximity to a patient's body may be utilized and that, in some embodiments, a blood pressure measurement controller 120 may not be used at all. It should further be appreciated that the finger cuff 104 may be connected to a blood pressure measurement controller described herein, or a pressure generating and regulating system of any other kind, such as a conventional pressure generating and regulating system that is located remotely from the body of the patient (e.g., a pump 134 located remotely from a patient). Any kind of pressure generating and regulating system that can be used, including but not limited to the blood pressure measurement controller, may be described simply as a pressure generating and regulating system. As a further example, in some embodiments, there may be no blood pressure measurement controller, at all, and a remote pump 134 that is controlled remotely may be directly connected via a tube 136 and 123 to finger cuff 104 to provide pneumatic pressure to the finger cuff 104.

During volume clamp measurements on the finger, the volume of the finger under the cuff decreases slowly as blood and interstitial fluids are pressed away. As a result, the tightness of the cuff may change and the cuff may become too loose. Measurements have shown that if the circumference of the finger is decreased by 3%, it affects reported blood pressure values substantially. This change in volume is especially the case in subjects with edema, in pregnant women, or when long term (e.g., 8 hour) measurements are made on the same finger.

Known methods for automatically assessing the application and/or tightness of the cuff are based on the measured pressure response of the cuff to quick inflation. These known methods can be performed only when the blood pressure measurement is not ongoing. Further, the known methods are susceptible to the influence of the resistance of the pneumatic tube 123 and therefore may be inaccurate. Also, it should be appreciated that although a finger cuff example is provided, embodiments of the invention to be hereafter described may be applied to other cuffs for other body parts, such as, arms, legs, etc.

A finger cuff 104 may comprise: a flexible printed circuit, an inflatable bladder, which, at a back-layer, may be attached to the flexible printed circuit, and an LED-PD pair. The flexible printed circuit may be electrically connectable to a cable provided with a suitable electric connector. Further, the flexible printed circuit may comprise a module for processing the signal from at least the photodiode.

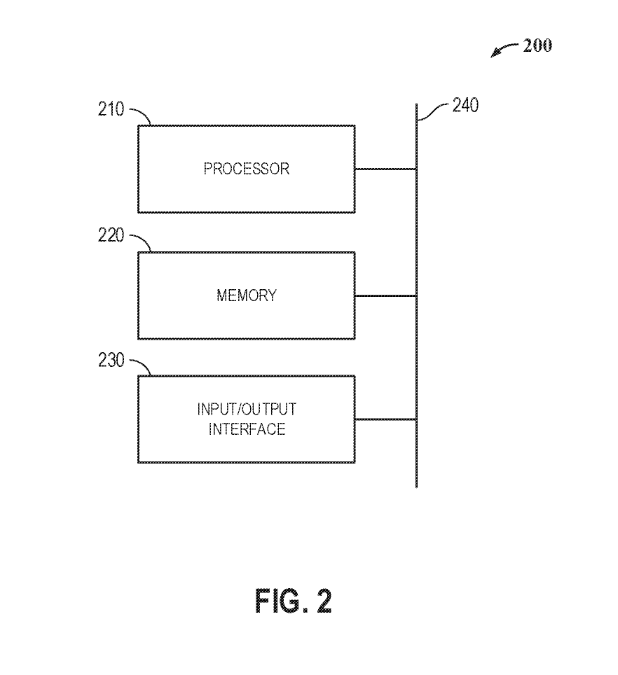

Referring to FIG. 2, a block diagram illustrating example control circuitry 200 is shown. The control circuitry 200 may correspond to the circuitry of one or more of: the blood pressure measurement controller 120, the patient monitoring device 130, other control circuitry that resides in the blood pressure measurement system 102, as appropriate, or any combination thereof. It should be appreciated that FIG. 2 illustrates a non-limiting example of a control circuitry 200 implementation. Other implementations of the control circuitry 200 not shown in FIG. 2 are also possible. The control circuitry 200 may comprise a processor 210, a memory 220, and an input/output interface 230 connected with a bus 240. Under the control of the processor 210, data may be received from an external source through the input/output interface 230 and stored in the memory 220, and/or may be transmitted from the memory 220 to an external destination through the input/output interface 230. The processor 210 may process, add, remove, change, or otherwise manipulate data stored in the memory 220. Further, code may be stored in the memory 220. The code, when executed by the processor 210, may cause the processor 210 to perform operations relating to data manipulation and/or transmission and/or any other possible operations.

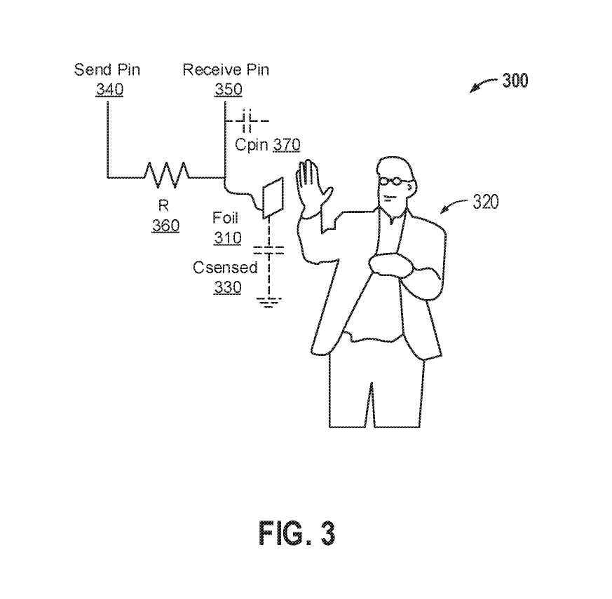

Referring to FIG. 3, a diagram illustrating an example cuff application/tightness assessment module 300 is shown. The module 300 may comprise a metal foil 310, which may be embedded in the finger cuff 104. It should be appreciated that the metal foil 310 may be replaced with any conducting component. For example, in a different embodiment, the conducting component may be a dedicated conducting region implemented on the flexible printed circuit of the finger cuff 104. As the metal foil 310 is embedded in the finger cuff 104, it can be assumed to be electrically insulated from the subject (patient) 320. The metal foil 310 is also electrically insulated from earth (i.e., ground (GND)) and from other electrical parts of the finger cuff 104. It should be appreciated that the presence of a body part (e.g., a finger) of the subject 320 near the metal foil 310 changes the capacitance value between the metal foil 310 and earth (e.g., C_{sensed} 330) because the subject's body is a conducting object. Further, the capacitance value between the metal foil 310 and earth increases as the distance between the body part and the metal foil 310 decreases. Therefore, the application and/or tightness of the cuff may be assessed indirectly based on the capacitance between the metal foil 310 and earth. Any known method for measuring capacitance can be utilized to measure the capacitance between the metal foil 310 and earth.

In one embodiment, the capacitance between the metal foil 310 and earth may be measured based on the charging time. In one simple implementation as illustrated in FIG. 3, a simple circuitry comprising a send pin 340, a receive pin 330, and a resistor 360 may be used to measure the capacitance between the metal foil 310 and earth. The send pin 340 and the receive pin 350 may be electrically connected via the resistor 360. Further, the metal foil 310 may be electrically connected to the receive pin 350.

To measure the capacitance between the metal foil 310 and earth (i.e., GND), a signal may be provided via the send pin 340 to charge the metal foil 310 with a known voltage or current. A person skilled in the art would understand that the charging time is a function of the capacitance between the metal foil 310 and earth. Therefore, the capacitance between the metal foil 310 and earth may be measured based on a time it takes for the voltage on the receive pin 350 to reach a predetermined threshold voltage after the charging started. It should be appreciated that the charging time is also influence by the parasitic capacitance Cₚᵣₑ 370. However, as the parasitic capacitance Cₚᵣₑ 370 can be assumed to be relatively constant, reliable measurements of the capacitance between the metal foil 310 and earth based on the charging time, as described above, are still possible.

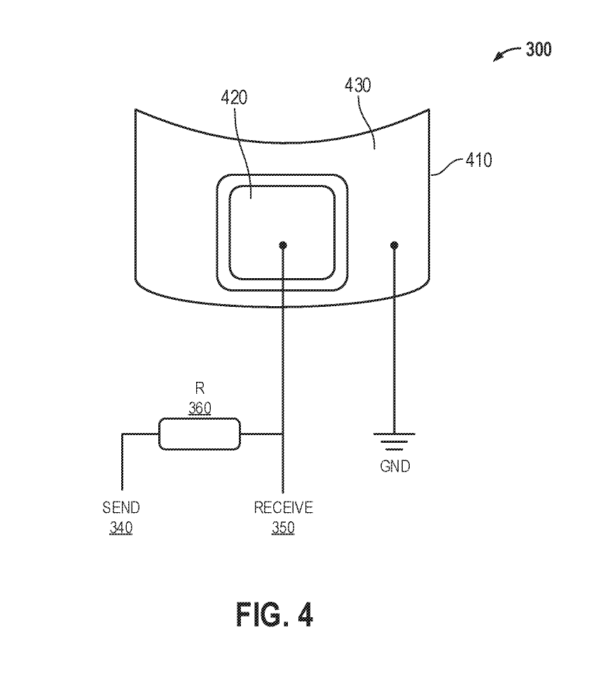

Referring to FIG. 4, a diagram illustrating an example cuff application/tightness assessment module 300 implemented in a flexible printed circuit is shown. It should be appreciated that only a relevant portion of the flexible printed circuit 410 is shown in FIG. 4. The flexible printed circuit 410 may comprise a first conducting region 420, which corresponds to the metal foil 310 of FIG. 3, and a second conducting region 430, which is electrically connected to earth (GND). The first conducting region 420 and the second conducting region 430 may be electrically insulated. Further, the first conducting region 420 may be electrically connected to the receive pin 350 directly, and may be electrically connected to the send pin 340 via the resistor 360.

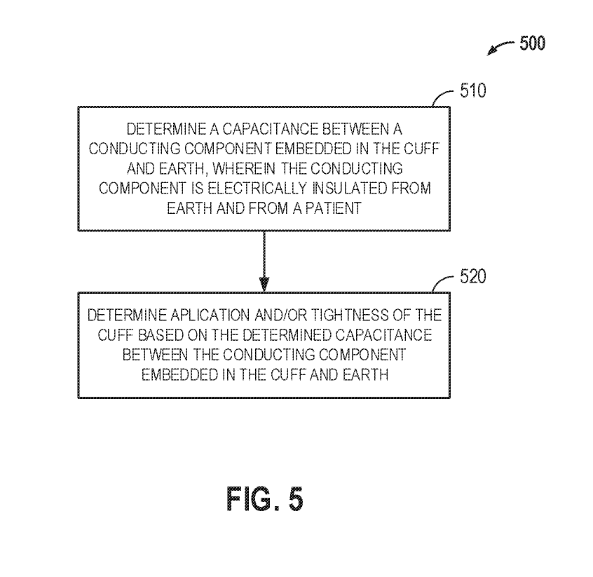

Referring to FIG. 5, a flowchart illustrating an example method 500 for assessing application and/or tightness of a cuff is shown. At block 510, a capacitance between a conducting component embedded in the cuff and earth may be determined, wherein the conducting component is electrically insulated from earth and from a patient. The conducting component may be a conducting region implemented on a flexible printed circuit of the cuff. The capacitance may be determined based on a charging time associated with the conducting component. The charging time may be measured with a send pin and a receive pin, wherein the conducting component is electrically connected to the receive pin directly, and electrically connected to the send pin via a resistor. The conducting component may be charged with a known voltage or current through the send pin. The charging time may be measured based on a time it takes for a voltage on the receive pin to reach a predetermined threshold voltage after charging started. At block 520, application and/or tightness of the cuff may be determined based on the determined capacitance between the conducting component of the cuff and earth. It should be appreciated that method 500 for assessing cuff application and/or tightness may be repeated from time to time as needed.

In a further embodiment, a plurality of cuff application/tightness assessment modules described above may be implemented within a single cuff. With additional assessment modules, application/tightness assessment may be more robust and/or versatile. For example, unusually large knuckles may be detected. In other words, a plurality of conducting components may be embedded in the cuff, and the capacitance associated with each conducting component may be independently determined to determine the application and/or tightness of the cuff.

As an example, it should be appreciated that control circuitry 200 including a processor 210, memory 220, and input/output interfaces 230 may be utilized to implement embodiments of the invention. For example, as previously described, a cuff for assessing application and/or tightness of the cuff may comprise: a conducting component; and a processor 210, the processor 210 to: determine a capacitance between the conducting component embedded in the cuff and earth, wherein the conducting component is electrically insulated from earth and from a patient; and determine application and/or tightness of the cuff based on the determined capacitance between the conducting component embedded in the cuff and earth.

Therefore, embodiments of the invention provide a method for assessing application and/or application of a cuff (e.g., a finger cuff, an arm cuff, a leg cuff, etc.) based on the capacitance between a conducting component embedded in the cuff and earth. The conducting component is a dedicated component that is electrically insulated from both earth and the subject. The capacitance increases as the distance between a body part of the subject and the conducting component decreases. Since during volume clamp measurements on the finger, the volume of the finger under the cuff decreases slowly (blood and interstitial fluids are pressed away), the tightness of the cuff changes. With a capacitive assessment method this can be monitored in real time and during a measurement. The method is especially sensitive over a wide range of finger-capacitive component distances. In other words, in addition to the presence of a finger or other body parts in the cuff, tightness of the cuff can also be detected. Because the presence of a finger (or other body parts, as appropriate) in the cuff can be sensed at any moment, more information may become available to the blood pressure monitor to guide end-users and improve the accuracy of blood pressure measurements. Further, premature unwrapping of the cuff during a measurement can be detected, and the measurement can be stopped.

It should be appreciated that aspects of the invention previously described may be implemented in conjunction with the execution of instructions by processors, circuitry, controllers, control circuitry, etc. (e.g., processor 210 of FIG. 2). As an example, control circuitry may operate under the control of a program, algorithm, routine, or the execution of instructions to execute methods or processes (e.g., method 500 of FIG. 5) in accordance with embodiments of the invention previously described. For example, such a program may be implemented in firmware or software (e.g. stored in memory and/or other locations) and may be implemented by processors, control circuitry, and/or other circuitry, these terms being utilized interchangeably. Further, it should be appreciated that the terms processor, microprocessor, circuitry, control circuitry, circuit board, controller, microcontroller, etc., refer to any type of logic or circuitry capable of executing logic, commands, instructions, software, firmware, functionality, etc., which may be utilized to execute embodiments of the invention.

The various illustrative logical blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A method for assessing application and/or tightness of a cuff, comprising:
determining a capacitance between a conducting component embedded in the cuff and earth, wherein the conducting component is electrically insulated from earth and from a patient; and
determining application and/or tightness of the cuff based on the determined capacitance between the conducting component embedded in the cuff and earth.

2. The method of claim 1, wherein the cuff is one of: a finger cuff, an arm cuff, or a leg cuff.

3. The method of claim 1, wherein the conducting component is a conducting region implemented on a flexible printed circuit of the cuff.

4. The method of claim 1, wherein the capacitance is determined based on a charging time associated with the conducting component.

5. The method of claim 4, wherein the charging time is measured with a send pin and a receive pin, and wherein the conducting component is electrically connected to the receive pin directly, and electrically connected to the send pin via a resistor

6. The method of claim 5, wherein the conducting component is charged with a known voltage or current through the send pin, and wherein the charging time is measured based on a time it takes for a voltage on the receive pin to reach a predetermined threshold voltage after charging started.

7. The method of claim 1, wherein a plurality of conducting components are embedded in the cuff, and the capacitance associated with each conducting component is independently determined to determine the application and/or tightness of the cuff.

8. A cuff for assessing application and/or tightness of the cuff comprising:
a conducting component; and
a processor, the processor operable to:
determine a capacitance between the conducting component embedded in the cuff and earth, wherein the conducting component is electrically insulated from earth and from a patient; and
determine application and/or tightness of the cuff based on the determined capacitance between the conducting component embedded in the cuff and earth.

9. The cuff of claim 8, wherein the cuff is one of: a finger cuff, an arm cuff, or a leg cuff.

10. The cuff of claim 8, wherein the conducting component is a conducting region implemented on a flexible printed circuit of the cuff.

11. The cuff of claim 8, wherein the capacitance is determined based on a charging time associated with the conducting component.

12. The cuff of claim 11, wherein the charging time is measured with a send pin and a receive pin, and wherein the conducting component is electrically connected to the receive pin directly, and electrically connected to the send pin via a resistor

13. The cuff of claim 12, wherein the conducting component is charged with a known voltage or current through the send pin, and wherein the charging time is measured based on a time it takes for a voltage on the receive pin to reach a predetermined threshold voltage after charging started.

14. The cuff of claim 8, wherein a plurality of conducting components are embedded in the cuff, and the capacitance associated with each conducting component is independently determined to determine the application and/or tightness of the cuff.
